(19) United States
(12) Patent Application Publication
(10) Pub. No.: US 2022/0296844 A1
(43) Pub. Date: Sep. 22, 2022

(54) PORTABLE AND COMPACT SYSTEM FOR DELIVERY OF HUMIDIFIED HIGH FLOW NASAL CANNULA (HHFNC) THERAPY IN NEONATES AND INFANTS

(71) Applicant: YALE UNIVERSITY, New Haven, CT (US)

(72) Inventor: Anjelica Gonzalez, New Haven, CT (US)

(21) Appl. No.: 17/637,100

(22) PCT Filed: Aug. 24, 2020
(86) PCT No.: PCT/US20/47600

§ 371 Date: Feb. 22, 2022

Related U.S. Application Data
(60) Provisional application No. 62/890,745, filed on Aug. 23, 2019.

(51) Int. Cl. A61M 16/10; A61M 16/12; A61M 16/06; A61M 16/00; A61L 9/20; G16H 20/40; H05B 3/54

(52) U.S. Cl. CPC A61M 16/109; A61M 16/1005; A61M 16/125; A61M 16/0672; A61M 16/0096; A61L 9/20; G16H 20/40; H05B 3/54; A61M 2205/36; A61M 2205/3653; A61M 2205/3368; A61M 2016/0033; A61M 2240/00; A61L 2209/12; H05B 2203/014

(57) ABSTRACT

A portable system for delivery of humidified high flow nasal cannula therapy includes a portable system housing, a respiratory circuit positioned within the housing comprising an ambient air conduit and an oxygen conduit merging downstream into a blended air conduit, an air pump positioned within the housing and connected to an upstream end of the ambient air conduit, configured to transfer ambient air into the ambient air conduit, an oxygen supply connection element connected to an upstream end of the oxygen conduit and configured to connect to an oxygen supply, a humidification chamber connected to a downstream end of the blended air conduit and an upstream end of an air output conduit, and a humidification chamber seating portion connected to the housing and configured to seat the humidification chamber, where the humidification chamber seating portion comprises a sterilization light source.

### CROSS-REFERENCE TO RELATED APPLICATIONS

This application claims priority to U.S. provisional application No. 62/890,745 filed on Aug. 23, 2019 incorporated herein by reference in its entirety.

### BACKGROUND OF THE INVENTION

Respiratory support devices are commonplace in hospitals around the world and are a significant medical and engineering advancement to improve pulmonary function. Their widespread adoption and implementation has saved thousands of lives and promise to continue to do so as advances in engineering technologies improve our ability to support physiological systems. Respiratory therapy has been particularly important for neonatal and infant patients up to one year of age, as they tend to be especially vulnerable and rely on respiratory aids to keep their airways open, regulate blood oxygen content, and maintain lung pressure to promote recovery (see e.g. Breathing Support For Premature Babies”. Tommys.org. N.p., 2016. Web. 10 Mar. 2016).

One of the most important advancements in this field to date has been the development of humidified high flow nasal cannula therapy (HHFNC), especially in the neonatal and pediatric context. HHFNC therapy is a non-invasive form of respiratory support that avoids the patient positioning difficulties, nasal trauma and agitation that can occur with traditional positive-pressure therapy devices (see e.g. “Breathing Support For Premature Babies”. Tommys.org. N.p., 2016. Web. 10 Mar. 2016). According to UNICEF, respiratory insufficiencies in neonates related to pre-term births and pneumonia account for nearly 30% of all neonatal deaths (see e.g. Chowdhury, Hafizur Rahman et al. “Causes of Neonatal Deaths in a Rural Subdistrict of Bangladesh: Implications for Intervention.” Journal of Health, Population, and Nutrition 28.4 (2010): 375-382).

Humidified high-flow nasal cannula therapy is an example of a life-saving medical technology that aims to provide high flows of humidified, warmed, and oxygenated air to keep lungs inflated during respiratory distress. Humidification is a key advantage of HHFNC systems because they prevent airway desiccation and discomfort, allowing infants to be kept under support for longer periods of time. Such devices can treat premature infants who suffer from a lack of surfactant and require external support to keep their alveolar sacs inflated. Infant HHFNC therapy has been widely taken up in neonatal intensive care units (NICUs) and pediatric intensive care units (PICUs) in hospitals in the United States and in other high-income settings. However, these pieces of capital equipment can be prohibitively expensive for NICUs, PICUs and hospitals in resource-poor settings and developing countries, restricting access to safe, effective care to infants around the world.

However, existing humidified high-flow nasal cannula respiratory aid devices are largely immobile and impractical for patients who are in transport or reside in difficult-to-reach, resource-poor settings. Existing devices rely on external infrastructure for operation, typically in hospital or clinical settings, and depend on outside sources of pressurized air, oxygen, sterilized water to humidify the airflow, as well as external power supplies. These external requirements for operation, combined with the lack of a mobile and compact housing, further restrict movement for patients, doctors, nurses and respiratory therapists during treatment. Furthermore, devices relying on external infrastructure could result in interrupted treatment in cases of power loss, and stationary devices relying on outlets do not allow for routine patient transports. HHFNC respiratory therapy in these cases would be cut short to move patients to an alternative but mobile treatment form that lacks the advantages of the HHFNC systems described above.

Thus, what is needed in the art is a portable and compact HHFNC system that can generate and deliver sterilized, humidified, warmed and oxygenated air to neonatal or infant patients by way of an extubated nasal cannula.

### SUMMARY OF THE INVENTION

In one embodiment, a portable system for delivery of humidified high flow nasal cannula therapy includes a portable system housing; a respiratory circuit positioned within the housing including an ambient air conduit and an oxygen conduit merging downstream into a blended air conduit; an air pump positioned within the housing and connected to an upstream end of the ambient air conduit, configured to transfer ambient air into the ambient air conduit; an oxygen supply connection element connected to an upstream end of the oxygen conduit and configured to connect to an oxygen supply; a humidification chamber connected to a downstream end of the blended air conduit and an upstream end of an air output conduit; and a humidification chamber seating portion connected to the housing and configured to seat the humidification chamber, wherein the humidification chamber seating portion includes a sterilization light source. In one embodiment, the system includes a first heat source positioned near the humidification chamber and configured to heat water stored in the humidification chamber. In one embodiment, the first heat source includes a heating blanket. In one embodiment, the heating blanket is configured to secure to and wrap around the humidification chamber when the humidification chamber is seated, and detach from the humidification chamber for removal of the humidification chamber. In one embodiment, the first heat source includes a heating wire. In one embodiment, the system includes a second heat source configured to heat the air output conduit. In one embodiment, the second heat source includes a heating wire. In one embodiment, the sterilization light source is a UV light. In one embodiment, the system includes a controller electrically coupled to a power supply, the air pump and at least one heating element. In one embodiment, the system includes at least one temperature sensor electrically coupled to the power supply, wherein the controller is configured to change a function of the air pump or at least one heating element based on a signal detected from the at least one temperature sensor. In one embodiment, the system includes at least one airflow sensor electrically coupled to the power supply, wherein the controller is configured to change a function of the air pump or at least one heating element based on a signal detected from the at least one airflow sensor. In one embodiment, the controller and power supply are positioned within the housing. In one embodiment, the humidification chamber is removable from the seating portion. In one embodiment, the seating portion includes a recessed external surface of the housing. In one embodiment, the housing includes a movable portion that is configured to open and close access to internal portions of the housing. In one embodiment, seating portion is configured on an external side of the movable portion. In one embodiment, the movable portion is connected to the housing by at least one hinge. In one embodiment, the sterilization light source is positioned in an upper cap portion of the seating portion. In one embodiment, the humidification chamber seating portion is positioned within the housing. In one embodiment, the system includes a nasal cannula connected to a downstream end of the air output conduit.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1A is a partial cutaway view of a portable HHFNC system showing interior components according to one embodiment; and FIG. 1B is a side view of the exterior of a portable HHFNC system according to one embodiment.

FIG. 2 is an isolated side view of a 3-way respiratory air circuit according to one embodiment.

FIG. 3A is an alternate side view of the exterior of a portable HHFNC system according to one embodiment; and FIG. 3B is an alternate partial cutaway view of a portable HHFNC system showing interior components according to one embodiment.

FIG. 4 is an isolated side view of an ultraviolet radiation component according to one embodiment.

FIG. 5 is a schematic diagram of a control circuit that controls power and current delivered to the pump, the heating elements and the ultraviolet radiation source according to one embodiment.

FIG. 6 is a system diagram of a portable HHFNC system according to one embodiment.

FIG. 7A is a perspective view of a system prototype and the heating blanket detached from the humidification chamber according to one embodiment; FIG. 7B is a perspective view of the heating blanket wrapped around the humidification chamber according to one embodiment; FIG. 7C is a magnified view of the sterilization light source according to one embodiment; FIG. 7D is a back-view of a hinged portion of the housing according to one embodiment; FIG. 7E is a partial internal view of the housing interior according to one embodiment; and FIG. 7F is an isolated view of a heating blanket.

### DETAILED DESCRIPTION OF THE INVENTION

It is to be understood that the figures and descriptions of the present invention have been simplified to illustrate elements that are relevant for a more clear comprehension of the present invention, while eliminating, for the purpose of clarity, many other elements found in systems for delivery of humidified high flow nasal cannula therapy. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein is a portable system for delivery of humidified high flow nasal cannula therapy.

The portable and battery-powered HHFNC system described herein allows for fully mobile infant patient care while delivering the necessary sterilized, humidified, warmed and oxygenated airflow outputs. Embodiments of the system includes components such as an internal battery source, heating sources for the water and tubing, an automated UV sterilization mechanism within the humidification chamber. In one embodiment, the water chamber is heated by a heating blanket which eliminates any temperature gradient in the water and allows for a uniform system output. The system components can be encased in a durable and compact external housing which protects the interior circuit and functional system components. The configuration of system components minimizes the space necessary for reliable generation of required airflow outputs, allowing for a system that is compact, handheld and easy to transport and store. Operation is simplified, and the system can be used with minimal training. By implementing a simple user interface, which in certain embodiments includes an on/off button and two flow rate control knobs for oxygen and total blended airflow, proper operation can be easily taught to clinicians, nurses, technicians and health aids. For purposes of portability, the battery power source allows for stationary use and recharging, with the option of running the system off a battery or externally-drawn power sources (e.g., wall outlets or sockets).

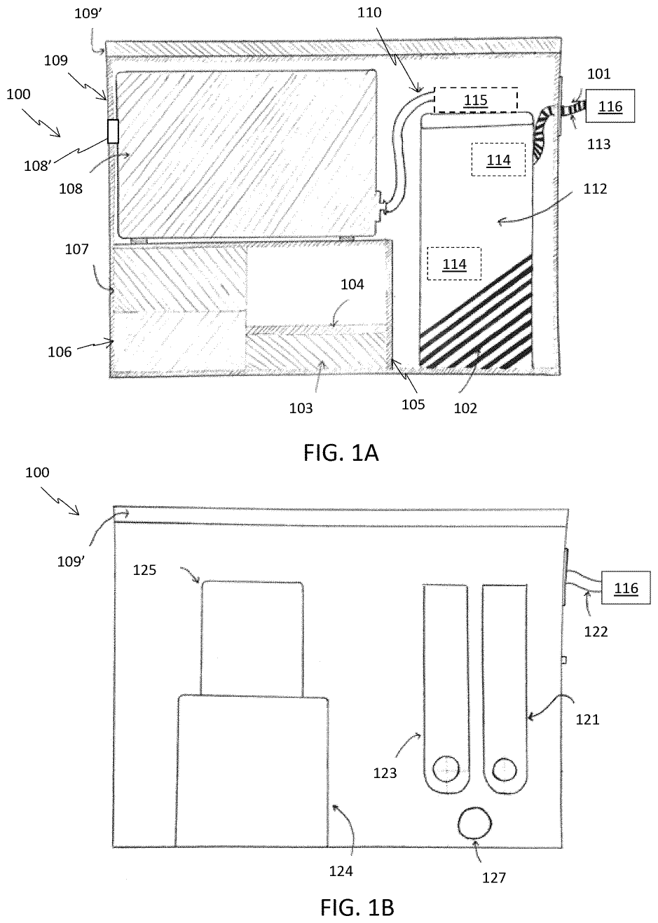

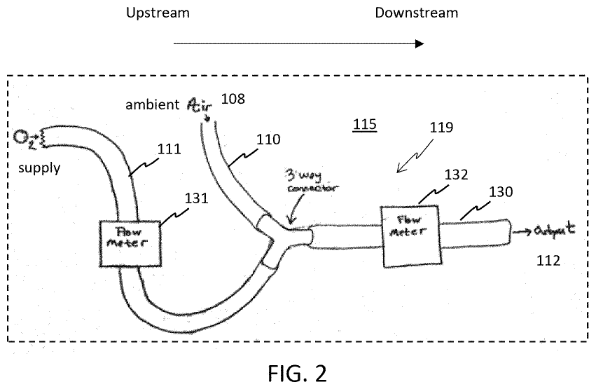

With reference now to FIG. 1A, according to one embodiment, the system 100 includes insulated respiratory air output tubing 101 with a wire heating element 113 that delivers the conditioned airflow to the patient. A separate heating element 102, is wrapped around the humidification chamber 112 to heat water stored in the humidification chamber 112 to a temperature of between 28-30 degrees Celsius. A controller 103, such as a printed circuit board is electrically coupled to components of the system 100 to regulate information transfer and power delivery to the electromechanical and sensor devices within the system 100. Various sensors 114 such as temperature sensors, humidity sensors, flowrate sensors and water-level sensors can placed in physical contact with components of the system, such as the humidification chamber 112 and distal end of the patient tubing 101 to provide continuous feedback to the controller 103 for regulating system operation 100, such as the temperature outputs of the resistive heating elements 102, 113 or manipulating air flow. A protective dividing layer 104 separates the controller 103 from other electromechanical and electronics components, including the power supply 106 (which in one embodiment is a lithium ion battery supply) and a power transformer 107. This layer 104 provides separation and protection of these components, prevents overheating of the controller 103 in proximity to the power supply 106, and allows for ease of insertion and removal by the user. The electronics housing 105 encases and protects the electronic components, and sits within a larger yet still portable and handheld system housing 109. The electronics housing 105 prevents exterior elements, including dust particles, insects or pests, and water from the humidification chamber, from interfering with the electronics operations. Similarly, the system housing 109 prevents exterior elements, including dust particles, insects or pests from interfering with or contaminating system components and the respiratory circuit 119, and maintains durability of the system. Interior mounts for both the electronics housing 105 and system housing 109 fix each component in place to prevent movement during operation and relocation. In one embodiment, controller and/or power and electronic components are separated into a separate box and connected to the system for communicating with and controlling the electromechanical components. A diaphragm pump 108 coupled to the controller 103 entrains room air through an ambient air inlet 108, such as the rapid reciprocating action of a rubber, Teflon or thermoplastic diaphragm. The pump 108 brings ambient room air into the system's respiratory circuit 119 (see e.g. FIG. 2), blending it with oxygen and moving the blended oxygen and room air into the humidification chamber 112 for airflow humidification and warming before being delivered to the patient via a nasal cannula 116.

Specifically with reference to FIG. 2, the respiratory circuit 119 tubing arrangement and connections for the precise mechanical blending of oxygen and room air is shown. The circuit 119 shows the configuration in which airflow is blended from separate oxygen and ambient air sources and delivered to the humidification chamber 112, isolated from the housing and other components for explanatory purposes. Oxygen (O₂) enters the medical-grade, biofouling resistant respiratory tubing 111 via a standard connection to an external source, such as an oxygen canister or hospital tank, the flow rate of which is regulated by the user with a first flow meter 131, affixed to the primary user interface. Separately, room air is drawn into tubing 110 of the circuit 119 by the pump 108, and the oxygen and entrained air turbulently mix at the 3-way connector junction. The rate of total blended airflow is user-regulated with the second flow meter 132, also displayed on the primary user interface. The desired oxygen concentration (between 21-100% O₂ concentration) of the mixed airflow can be precisely mechanically controlled by the user by adjusting the flow rate of the first oxygen flow meter in relation to the total blended flow rate set at the second flow meter 132. Taking a simple ratio of the total flow rate and the flow rate of oxygen, while incorporating a corrective term for the amount of oxygen in ambient air, determines the percent concentration of oxygen flow to the patient. This two-stage mixing and control design allows for varying combinations of total flow rate and percent oxygen therapy, with a typical volumetric flow rate range of between 0-10 liters per minute, and an oxygen concentration of between 21-100%, as deemed clinically necessary for neonatal or infant patients in need of this form of respiratory support. The blended oxygen and air output of this portion of the circuit is subsequently humidified and warmed for delivery to the neonatal or infant patient.

With reference back to FIG. 1B, the exterior, front-facing profile and primary user interface of the device are displayed according to one embodiment. The oxygen airflow meter of 121 regulates the volumetric flow rate of pressurized oxygen. The flow rate setting can be precisely adjusted in conjunction with the total airflow meter of 123 to achieve the desired oxygen concentration of mixed airflow delivered to the patient, according to a simple linear relationship. Insulated and heated respiratory tubing 101 is connected to a nasal cannula (or mask) 116 at the distal end of the circuit to deliver conditioned airflow to the patient. A sleeve or protruding support enclosure 124 holds the optional oxygen canister 125 in place, which supplies pressurized oxygen to the system. The device housing lid 109′ can be easily opened or shut to monitor, clean and replace interior components, as well as protect them from hazardous external conditions including water, dust, animal or pest contamination, or shaking or movement of the device, particularly during use in patient transport. The on/off button 127 is the central control to power on and off the device. Pressing once to turn the device can initiate both the automated UV sterilization of water in the humidification chamber (e.g. for 30-90 seconds), as well as the heating of the humidification chamber 112 and insulated respiratory tubing 101. After this period sterilizing the humidification chamber 112, the interior diaphragm pump 108 receives power and outputs entrained air through the respiratory circuit 119. The order of electromechanical operations and power delivery is controlled by pre-programmed wiring in the interior printed circuit board, described according to certain embodiments in more detail below.

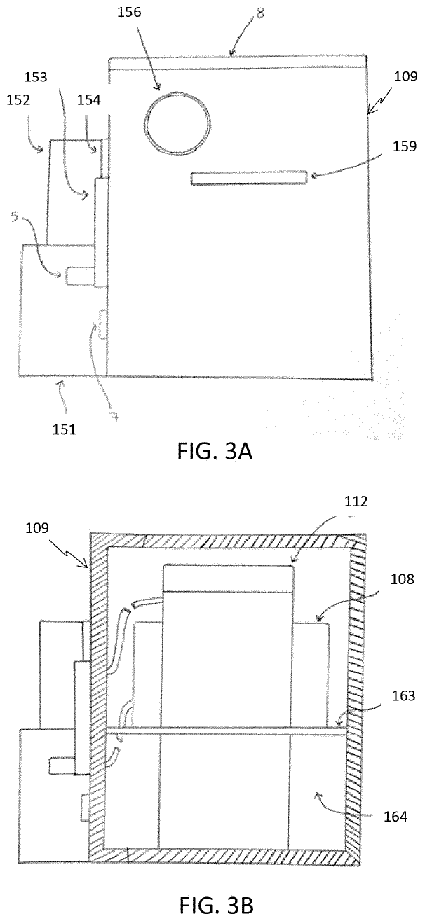

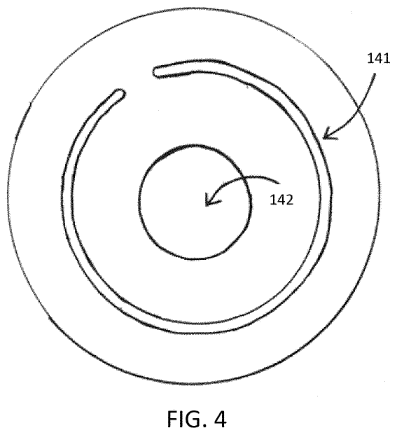

With reference now to FIGS. 3A and 3B (an alternate side view of the system 100), a protruding sleeve or holster 151 supports an oxygen canister 152 for the optional delivery of oxygen into the system 100. An oxygen airflow meter 153 represents one of multiple user controls during operation. The flow rate setting can be precisely adjusted by a dial, corresponding to a total airflow meter 154 of the same form and control mechanism, to achieve the desired flow rate and oxygen concentration of air delivered to the patient. An opening 156 for the insulated, heated respiratory tubing provides an exit for the heated respiratory conduit to deliver the conditioned gas to the neonatal patient through a nasal cannula interface connected at the distal end of the respiratory tubing. The on/off button 127 is one of multiple controls on the primary interface. In one embodiment, pressing this once powers on the device, initiating both the automated UV sterilization of water in the humidification chamber 112, and the heating of the humidification chamber and heating respiratory tubing. After a sufficient period of time to sterilize the liquid in the humidification chamber 112, typically between 30-90 seconds, the interior diaphragm pump 108 is switched on to begin delivering entrained air through the respiratory circuit 119. This order of electromechanical operations is controlled by instructions programmed into the controller 103. The device housing lid 109′ is easily opened or shut to monitor, clean and replace interior components, as well as protect these components from hazardous external conditions including water, dust, animal or pest contamination, shaking, or movement of the device (for example during use in patient transport). The system handle 159 provides ease of lifting and transport of the device, in one embodiment on the exterior left wall of the system. In certain embodiments, the handle can be positioned on the exterior top of the housing lid 109′, or on opposing walls of the housing 109. Optional wheels can be added onto portions of the design to aid with device transport, and alleviate the full weight of the device for the user during transport.

Referring specifically now to FIG. 3B, the diaphragm pump 108 entrains room air into the respiratory circuit, through a HEPA or otherwise suitable medical-grade air filter. It is capable of delivering the full suitable range of volumetric airflow rates for neonatal and infant patients, typically between 0-10 liters per minute up to a maximum of 30 liters per minute. The cap for the humidification chamber 112 contains an ultraviolet radiation source on its interior underside, for the automated sterilization of the air and water contents of the chamber. This component is described in further detail in reference to FIG. 4. A stabilizing support divider 163 holds the diaphragm pump 108, humidification chamber 112 and electronics housing 164 in place within the interior. The support divider 163 provides stabilization for the primary interior device components, particularly during portable use. The electronics exterior housing 164 encases and protects the electronics components, including the controller, power source and power transformer. This housing prevents exterior elements, such as dust particles, insects or pests, and water from the humidification chamber, from contaminating or interfering with electronics operations. Medical-grade tubing (such as Non-Phthalate Polyvinyl Chloride (PVC) Tubing), provides a conduit for the blended oxygen and room air to travel into the humidification chamber for airflow humidification and warming.

With reference back to FIGS. 1A and 2, during operation, the blended oxygen and entrained airflow enters the humidification chamber 112 through the medical grade tubing 130, which is partially filled with water (in one embodiment, between 200-300 milliliters, within a full range of 0-500 milliliters). The blended airflow is bubbled directly in the water, which in this contained, warmed environment reaches full saturation. The humidification chamber 112 also serves as the location for the automated ultraviolet sterilization of the water.

In one embodiment, a UV-C sterilization source 142 is located on the interior underside of the cap of a seating portion of the humidification chamber. The UV-C light 142 is separated from the water chamber by a UV-C permeable surface that allows the full strength of the generated UV light to disinfect the water. Referring in more detail to FIG. 4, an embodiment of the UV radiation apparatus is shown in isolation, located on the interior, underside of the lid of the humidification chamber 112. The circular UV light fixture 141 emits electromagnetic radiation at a wavelength between 100-400 nanometers. Ultraviolet radiation of this directed nature and duration (between 30-90 seconds, immediately after the device has been switched on) has demonstrated sufficient efficacy in medical literature and experimental verification the sterilization of harmful bacteria (e.g., Escherichia coli, Pseudomonas) to the newborn patient, within a simple cylindrical container of relatively small volume (between 0 to 500 milliliters). The initiation and duration of radiation is controlled by the controller 103. The Light Emitting Diode (LED) 142 flashes once to indicate the initiation of the automated UV sterilization process after the device has been switched on, and twice at the end of the UV sterilization process. This LED information transmission process is complementary to the precise regulation of the ultraviolet radiation source 141 by the controller, and provides a secondary signal to the user that either the radiation process has begun or has completed, after the device has been switched on.

The UV sterilization stage is key to safe patient treatment and serves as an additional safety mechanism beyond the use of sterile water, neutralizing or preventing the formation of harmful microbes at-source or prior to airflow entry into the humidification chamber. Simultaneously, during this stage of the humidification process, the heat source 102 in thermal contact with the humidification chamber warms the environment such that the interior temperature of the fully vapor saturated, mixed gas in the chamber above the water level reaches the range of 26-30 degrees Celsius. The fully saturated gas above water level exits the humidification chamber through the insulated, heated respiratory tubing 101 for delivery to the neonatal patient. A second conductive heating element 113 such as a heated resistance wire (e.g., an industrial heating cable), is contained within a support sleeve and wrapped around the medical-grade respiratory tubing 101 at the distal, patient-end of the circuit. In one embodiment, the temperature of this resistive heating element is set to vary between 31-34 degrees Celsius, above that of the humidification chamber, thus lowering the relative humidity of the airstream to below 100%. This is beneficial to mitigate the potential for condensation and the adverse effects of water droplet inhalation on the patient.

The flow of heat to the different parts of the system is monitored in a non-invasive manner by taking advantage of the finite heat loss across the conducting surfaces and the well-mixed condition of turbulent flows. In one embodiment, two or more temperature sensors 114 are placed on the external surface of the humidification chamber 112 and respiratory tubing 101 to capture an average temperature over the entire surface. These sensors 114 provide temperature feedback to the controller 103. The controller 103 regulates the power output and temperature of the corresponding heating elements 102, 113. The respiratory tubing 101 remains heated through the distal end of the circuit and entrance to the nasal cannula, reducing the likelihood of rain-out. The conduction and convection of this heating element raise the temperature of the conditioned airflow to 31-34 degrees Celsius, lowering the relative humidity of the airflow to between 90-95%, as required for optimal neonatal patient treatment. This compact system 100 contained within a single housing 109 takes advantage of the finite heat loss across the conducting surfaces of its components, and the well-mixed condition of turbulent flows to efficiently regulate the required temperatures to produce warmed and humidified air. The system further minimizes the amount of power required to maintain warmed and humidified air by recapturing ambient heat loss, generating the desired air output without requiring a complex power source, all within a compact system that is low cost and portable. For these same reasons and based on configurations of the system described herein, the system allows for the automatic, efficient and simultaneous sterilization and heating of humidified air in the respiratory circuit. Embodiments of the system can be implemented in field environments (e.g. developing countries, rural or impoverished areas) that lack the infrastructure to support larger and complex systems that are typically only found in state of the art hospitals.

In one embodiment, the heating element 102 is a conductive heating blanket that is wrapped uniformly around the entirety of the humidification water chamber 112. The heating blanket is uniformly wrapped to eliminate temperature gradients in the water that might be present by other methods, such as a bottom hot plate. In one embodiment, the heating blanket includes a thermal blanket with 3/16 in foam strips inserted into pleats, intended to help keep the blanket vertical and maintain structural integrity. In one embodiment, the heating element 102 is a heated resistance wire (e.g., an industrial heating cable) wrapped around the humidification water chamber 112. In one embodiment, a heated resistance wire can be fixed within a support sleeve or other separation layer, allowing for ease of humidification chamber removal for cleaning and re-filling.

In one embodiment, the pump 108 generates the positive displacement of air by repeatedly drawing in atmospheric particles through volumetric expansion and compression of the diaphragm. Other types of pumps or air blowers that do not utilize this diaphragm mechanism to generate positive airflow displacement can also be used. The pump 108 is capable of delivering the full range of desired or appropriate volumetric airflow rates for neonatal patients: typically between 0 to 10 liters per minute, and up to a maximum of 30 liters per minute. A filter—preferably a High Efficiency Particulate Arrestance (HEPA) or other suitable medical-grade filters—at the entrance of the pump blocks the suction of undesirable particulates, such as, but not limited to, dust mites and pollen. The filter in the one embodiment has dimensions of 3.5″×0.5″×3.0″, removes 99.97% particles that have a size of 0.3 μm, and can be easily washed or reused. This ensures the neonatal patient is not inhaling any irritants or foreign substances that could compromise the health, safety and efficacy of the infant's respiratory treatment.

In one embodiment, the power supply 106 is a lithium ion battery power supply which powers the electromechanical components of the device, including the UV sterilization lamp within the humidification chamber, the heated resistance wires heating the humidification chamber and distal respiratory tubing, and the diaphragm pump drawing and pushing entrained air through the circuit. The device is designed for regular, sustained operation from a rechargeable battery-pack or an external power source such as via a regular wall outlet. The power transformer 107 addresses the mismatch in voltage requirements for operation between the power source and other electromechanical components. The incoming Alternating Current (AC) power supply is diverted to an AC-DC (Direct Current) converter, and then DC power is supplied to the remaining parts of the device, namely, but not limited to, the heating elements, air pump, and electronic control circuitry. In one embodiment, it converts the voltage delivered by the lithium ion battery power supply 106 to that required to appropriately power the electromechanical elements of the device (e.g., from 220 volts alternating current to 110V alternating current), including the controller 103, diaphragm pump 108, heated resistance wires in 102 and 113, and the ultraviolet radiation source within the humidification chamber 112.

The device housing lid 109′ can be easily opened or shut to monitor, clean, and replace interior components, as well as protect these components from hazardous external conditions including water, dust, animal or pest contamination, shaking or movement of the device, for example during use in patient transport. Medical-grade tubing 110 & 111, such as but not limited to Non-Phthalate Polyvinyl Chloride (PVC) Tubing, provides a conduit for filtered room air drawn in and pumped out of the diaphragm pump 108 to mix with oxygen before entering the humidification chamber 112. The humidification chamber 112 in one embodiment is a simple plastic water bottle made of a material that maintains its integrity and mechanical and chemical properties when exposed to UV-C radiation and raised temperatures of up to 50 degrees Celsius (e.g., co-polyester-based Nalgene® water bottle). The primary function of the humidification chamber 112 is to humidify the incoming airstream to saturation at the dew point of the therapy set point (i.e., to 100% relative humidity at a temperature set point of 26-30 degrees Celsius). Humidification is achieved by the application of heat and the natural mixing properties of the incoming airstream, described in further detail below.

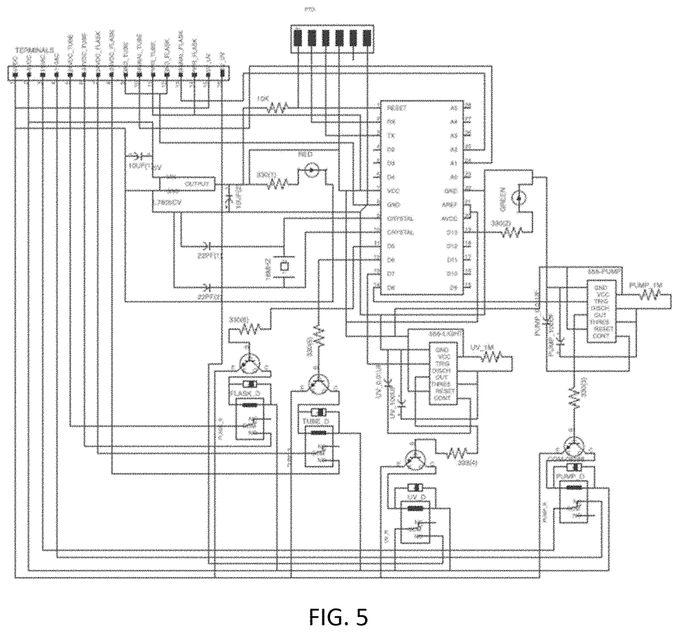

In one embodiment, certain parameters of the system can be preset by the controller 103 (e.g. simultaneous sterilization and heating for a predefined period, such as 30-90 seconds prior to pump airflow generation). Access to system parameters can be via a user interface attached to the system 100 or a communication module that can connect to a remote device such as a remote computer system or handheld mobile device. A GPS module can also be associated with the system to automatically adjust system setting based on parameters such as weather and regional characteristics. A schematic of the wiring of a controller circuit that electromechanically controls the power delivered to the pump, two heating elements, and UV radiation source is shown in FIG. 5 (for explanatory purposes, a high-level description of the wiring is provided). One primary circuit controls the aforementioned electromechanical processes, but can be broken down into discrete segments for each complementary function. The relays serve as automated on-off switches for the diaphragm pump, heating cables, and ultraviolet radiation source. Timers within the circuitry determine the order of component operation when the device is powered on. The UV-C radiation source and heating cables are first delivered power for 30-90 seconds, to initiate the sterilization and humidification chamber and distal tubing heating processes respectively. After this process is complete, the pump receives power and outputs airflow at the user-set total blended flow rate.

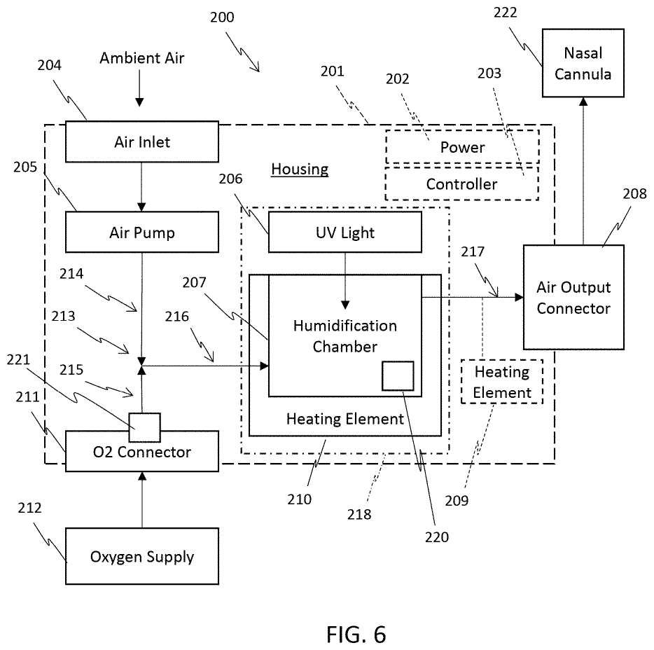

With reference now to the system diagram shown in FIG. 6, a portable system 200 for delivery of humidified high flow nasal cannula therapy is shown according to one embodiment. The system 200 includes a portable system housing 201 that houses a respiratory circuit 213, positioned within the housing 201. The respiratory circuit 214 includes an ambient air conduit 214 and an oxygen conduit 215 merging downstream into a blended air conduit 216. An air pump 205 is positioned within the housing 201 and is connected to an upstream end of the ambient air conduit 214. The air pump 205 is configured to transfer ambient air into the ambient air conduit 214 via an air inlet 204 positioned in an outer wall of the housing 201. An oxygen supply connection element 211 is also positioned in an outer wall of the housing 201, and is connected to an upstream end of the oxygen conduit 215 and configured to connect to an oxygen supply 212. The connection element 211 can be for example a tube connected to a valve on the oxygen supply, or a direct connection to the valve on a pressurized oxygen tank or canister. A humidification chamber 207 is connected to a downstream end of the blended air conduit 216 and an upstream end of an air output conduit 217. A humidification chamber seating portion 218 is connected to the housing 201 and configured to seat the humidification chamber 207. The humidification chamber seating portion 218 includes a sterilization light source 206.

In one embodiment, the system 200 includes a first heat source 210 positioned near the humidification chamber 207 and configured to heat water stored in the humidification chamber 207. In one embodiment, the first heat source 210 is a heating blanket and it's configured to secure to and wrap around the humidification chamber 207 when the humidification chamber 207 is seated. The heating blanket can also detach from the humidification chamber 207 for removal of the humidification chamber 207, such as during water refilling. In one embodiment, the first heat source 210 includes a heating wire. In one embodiment, the system 200 includes a second heat source 209 configured to heat the air output conduit 217. The air output conduit 217 in one embodiment can terminate in an air output connector 208, such as conventional connectors known in the art for connecting to a nasal cannula 219 or patient mask. In one embodiment, the second heat source 209 includes a heating wire. In one embodiment, the sterilization light source 206 is a UV light. In one embodiment, the system 200 includes a controller 203 electrically coupled to a power supply 202, the air pump 205 and at least one heating element 209, 210. In one embodiment, the system 200 includes at least one temperature sensor 220 electrically coupled to the power supply 202, and the controller 203 is configured to change a function of the air pump 205 or at least one heating element 209, 210 based on a signal detected from the at least one temperature sensor 220. In one embodiment, the system 200 includes at least one airflow sensor 221 electrically coupled to the power supply 202, and the controller 203 is configured to change a function of the air pump 205 or at least one heating element 209, 210 based on a signal detected from the at least one airflow sensor 221. In one embodiment, the controller 203 and power supply 202 are positioned within the housing 201. In one embodiment, at least one of the controller 203 and power supply 202 are in a separate housing and functionally connected to the electrical and electromechanical components in the system 200. For example, the system 200 can be controlled and monitored by a remote controller in communication with system components, and the remote controller may be cloud based or reside on a mobile handheld smart device. In one embodiment, the humidification chamber 207 is removable from the seating portion 218. In one embodiment, the humidification chamber seating portion 218 is positioned within the housing. In one embodiment, the system 200 includes a nasal cannula 222 connected to a downstream end of the air output conduit.

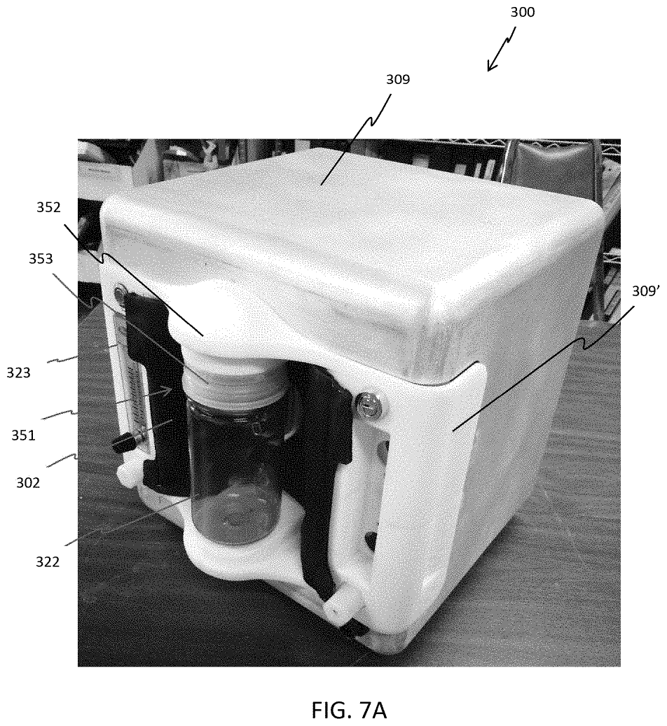

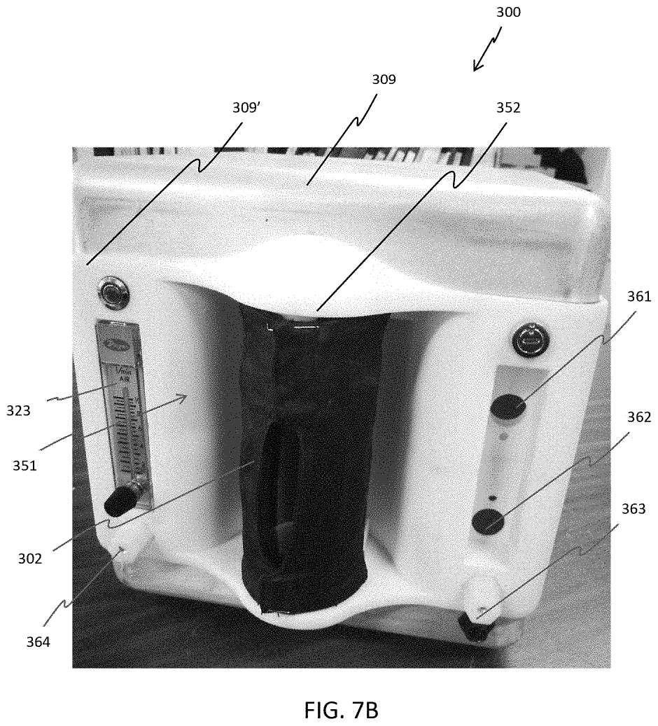

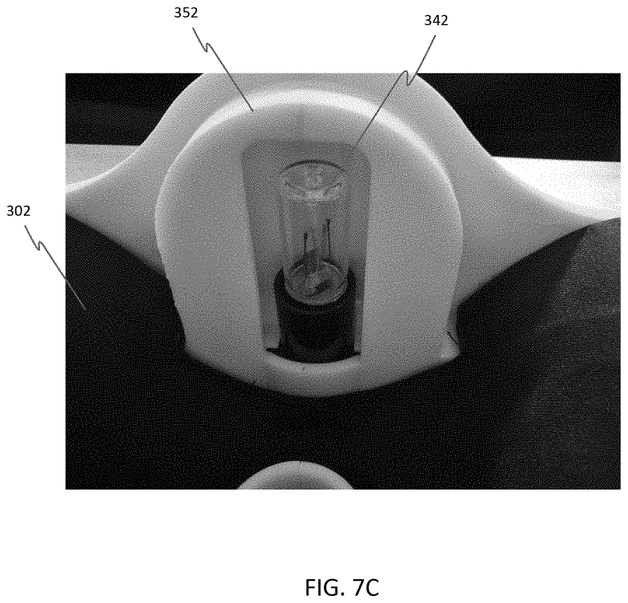

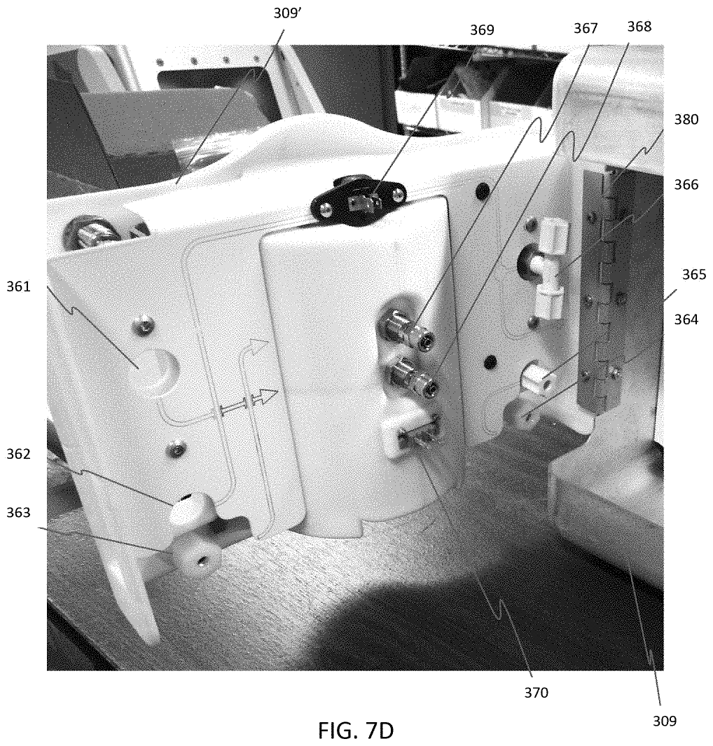

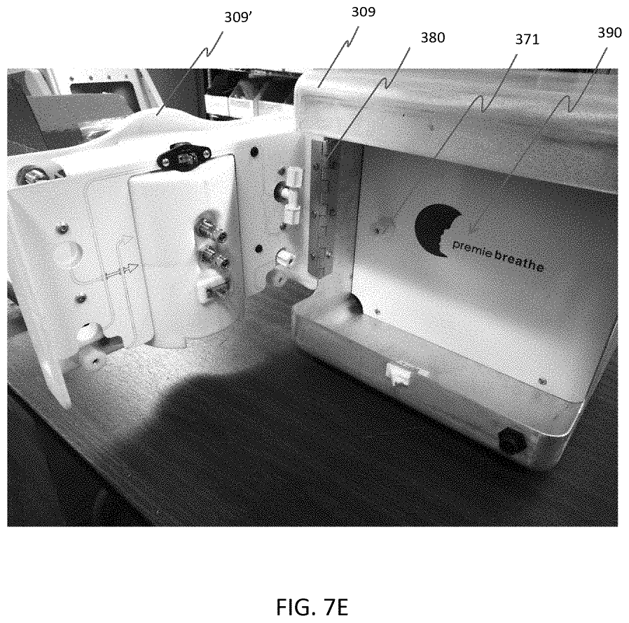

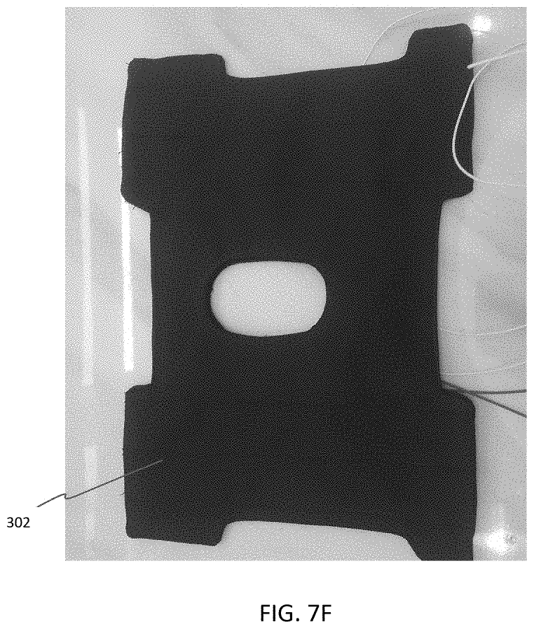

In one embodiment, the seating portion 218 includes a recessed external surface of the housing 201 (see e.g. the embodiment shown in FIGS. 7A-7E). In one embodiment, the housing 201 includes a movable portion that is configured to open and close access to internal portions of the housing 201. In one embodiment, the seating portion 218 is configured on an external side of the movable portion. In one embodiment, the movable portion is connected to the housing 201 by at least one hinge. In one embodiment, the sterilization light source 206 is positioned in an upper cap portion of the seating portion 218.

Embodiments of the systems and methods described herein can be utilized to help treat various conditions or supplement treatment therapies for neotates, including but not limited to asthma, bronchiolitis, obstructive sleep apnea syndrome, pneumonia, myopathies, ventilator management (weaning), patients with good respiratory drive but still needing minimal respiratory support, and providing malnourishment prevention if the neonate needs to be on respiratory support. In adults, embodiments of the systems and methods described herein can be utilized to help treat various conditions or supplement treatment therapies including but not limited to obstructive sleep apnea syndrome, chronic obstructive pulmonary disease with exacerbation, acute congestive heart failure with pulmonary edema, acute lung injury, pneumonia and ventilator weaning.

Embodiments of the system and method described herein have several advantages. The compact system contained within a single housing takes advantage of the finite heat loss across the conducting surfaces of its components, and the well-mixed condition of turbulent flows to efficiently regulate the required temperatures to produce warmed and humidified air. What typically happens in conventional systems is that they have to generate a lot more heat in a system to get the air and the water heated to the temperature needed to induce humidification. Embodiments of the system described herein, since it is compact and contained in a single housing, reduce the heat loss and take advantage of that energy generated to heat more efficiently. This is an advantage of configuring the system into a single compact housing. For example, traditional bubble cpap require two energy sources (some external) which requires an increase in energy, and energy is lost since it is not in a contained compact system or housing. Embodiments described take advantage of a configuration that keeps this heat internal and recycles it to induce humidification.

Additionally, embodiments described herein take advantage of a well-mixed condition of turbulent flows that contributes to efficiency for regulating the required temperatures to produce warmed and humidified air. Laminar flow in a system provides direct airflow without mixing, whereas turbulent air induces mixed air in a system, so you get an advantage of mixing the air in the direction of the flow. So for turbulent air, an oxygen and an ambient air input are provided that come in at two different temperatures, then they get mixed to the temperature desired. On the other hand, two separate laminar flows at the end if not mixed in the chamber end up having to mix outside or via an external mixer to get the desired temperature. So by having turbulent flow they are mixed in the system internally and this is more efficient with heat exchange. Thus, embodiments of the system described herein have regulators in the front to manage oxygen content by percent or ml that's all mixed in the heating portion so advantageously all that air is humidified. Conventional systems have ambient air humidified and an external O2, losing the advantage of having everything mixed at once (since one temperature is being diluted by the other temperature coming in). Conventional systems have the heating/humidification elements, the flow rate generator, and the O2 mixing external and have not provided a configuration into a single compact system or housing.

Still further, embodiments of the system allow for the automatic, efficient and simultaneous sterilization and heating of humidified air in the respiratory circuit. Sterilization sources can be for example IR to get through viral bacterial walls, or UV. Most conventional systems have been designed primarily for developed countries where there's no issue with clean water sources. For developing countries, the water being clean is a huge issue. Thus, embodiments advantageously provide a system that can be used in areas that have limits with regard to clean water, power, and require portability and simplicity.

In addition to the several advantages discussed above, embodiments of the system and method described herein have several advantages over traditional CPAPs and other conventional devices providing respiratory support. The embodiments described herein help to avoid complex implementation strategies, restrictions in patient positioning necessitated by CPAP treatment that can compromise therapeutic efficacy, and complications associated with CPAP use. Pressure-induced nasal trauma from CPAP prongs or masks is common in both HIC and LMICs, with rates highest among preterm and VLBW infants. Nasal tissue injury is painful, increases risk of infection, and in severe cases has long-lasting cosmetic implications. Gastric distention from CPAP (CPAP belly) can prevent infants from feeling hungry, resulting in malnutrition, while bulky tubing, and the need for a constant seal with nCPAP can interfere with breastfeeding and kangaroo mother care, two key components of newborn care plans.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

With reference now to FIGS. 7A-7F, a prototype system 300 is shown. First, with reference primarily to FIGS. 7A and 7B, the system 300 includes an external housing 309 with a hinged 380 cover portion 309′ that seats the humidification chamber 322. The cover portion 309′ opens and closes access to internal areas 390 of the housing 309. A heating blanket 302 lines an external recessed seating area 351 on the hinged cover portion 309 (shown isolated in FIG. 7F). When the system 300 is ready for use, the heating blanket 302 can be wrapped around the humidification chamber 322, which in certain embodiments is a simple plastic water bottle made of a material that maintains its integrity and mechanical and chemical properties when exposed to UV-C radiation and raised temperatures of up to 50 degrees Celsius (e.g., co-polyester-based Nalgene brand water bottle). An airflow meter 323 is positioned on the front of the hinged cover portion to set and monitor system airflow. A humidified air delivery tube output 364 is configured in the cover portion 309′. Tubing can be connected at this output for delivery of heated humidified air to the nasal cannula. Anchors 361, 362 for an O2 mixing regulator are configured in the cover portion 309′, replicating the air flow regulator 323. An O2 tank/concentrator connector 363 is configured in the cover portion 309′, for O2 input into the system. As shown specifically in FIG. 7C, a UV-C sterilization source 342 is located on the interior underside of the cap 252 of the humidification chamber 322, and within the external recessed seating area 351. The UV-C light 342 is separated from the water chamber 322 by a UV-C permeable surface 353 that allows the full strength of the generated UV light to disinfect the water. In the rear views of FIGS. 7D and 7E, elements include an air flow regulator anchored onto the unit 365, 366, an air valve for O2/air input into humidification chamber/water reservoir 367, an air valve for humidified O2/air output to the air flow regulator 368, UV light power 369, heating element power 370, and an ambient air inlet 271 (connects to 366 for mixing with O2). With reference specifically to FIG. 7F, the heating blanket 302 is made of cloth (vinyl or polyester) with foam inserts that insulate the heating wires. The heating blanket 302 is held in place via magnets in small pockets on the blanket flaps, that hold it closed when in place.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A portable system for delivery of humidified high flow nasal cannula therapy comprising:
a portable system housing;
a respiratory circuit positioned within the housing comprising an ambient air conduit and an oxygen conduit merging downstream into a blended air conduit;
an air pump positioned within the housing and connected to an upstream end of the ambient air conduit, configured to transfer ambient air into the ambient air conduit;
an oxygen supply connection element connected to an upstream end of the oxygen conduit and configured to connect to an oxygen supply;
a humidification chamber connected to a downstream end of the blended air conduit and an upstream end of an air output conduit; and
a humidification chamber seating portion connected to the housing and configured to seat the humidification chamber, comprising a sterilization light source.

2. The system of claim 1 further comprising:
a first heat source positioned near the humidification chamber and configured to heat water stored in the humidification chamber.

3. The system of claim 2, wherein the first heat source comprises a heating blanket.

4. The system of claim 3, wherein the heating blanket is configured to secure to and wrap around the humidification chamber when the humidification chamber is seated, and detach from the humidification chamber for removal of the humidification chamber.

5. The system of claim 1, wherein the first heat source comprises a heating wire.

6. The system of claim 1 further comprising:
a second heat source configured to heat the air output conduit.

7. The system of claim 6, wherein the second heat source comprises a heating wire.

8. The system of claim 1, wherein the sterilization light source is a UV light.

9. The system of claim 1 further comprising:
a controller electrically coupled to a power supply, the air pump and at least one heating element.

10. The system of claim 9 further comprising:
at least one temperature sensor electrically coupled to the power supply, wherein the controller is configured to change a function of the air pump or at least one heating element based on a signal detected from the at least one temperature sensor.

11. The system of claim 9 further comprising:
at least one airflow sensor electrically coupled to the power supply, wherein the controller is configured to change a function of the air pump or at least one heating element based on a signal detected from the at least one airflow sensor.

12. The system of claim 9, wherein the controller and power supply are positioned within the housing.

13. The system of claim 1, wherein the humidification chamber is removable from the seating portion.

14. The system of claim 13, wherein the seating portion comprises a recessed external surface of the housing.

15. The system of claim 13, wherein the housing comprises a movable portion that is configured to open and close access to internal portions of the housing.

16. The system of claim 15, wherein seating portion is configured on an external side of the movable portion.

17. The system of claim 16, wherein the movable portion is connected to the housing by at least one hinge.

18. The system of claim 16, wherein the sterilization light source is positioned in an upper cap portion of the seating portion.

19. The system of claim 1, wherein the humidification chamber seating portion is positioned within the housing.

20. The system of claim 1 further comprising:
a nasal cannula connected to a downstream end of the air output conduit.
